(19) United States
(12) Patent Application Publication
(10) Pub. No.: US 2019/0159760 A1
(43) Pub. Date: May 30, 2019

(54) PHOTOACOUSTIC PROBE

(71) Applicant: CANON KABUSHIKI KAISHA, Tokyo (JP)

(72) Inventor: Shinji Ohishi, Oyama-shi (JP)

(21) Appl. No.: 16/199,860

(22) Filed: Nov. 26, 2018

(30) Foreign Application Priority Data
Nov. 29, 2017 (JP) 2017-229202

(51) Int. Cl. A61B 8/00; G01N 29/24; A61B 8/15; A61B 5/00

(52) U.S. Cl. CPC A61B 8/4483; A61B 5/0095; A61B 8/15; G01N 29/2418

(57) ABSTRACT

A photoacoustic probe comprising: a receiving unit configured to receive photoacoustic waves generated from an object irradiated with light; a signal processing unit configured to perform a process for outputting photoacoustic signals originating from the photoacoustic waves; a communication unit configured to perform wireless communication with a photoacoustic apparatus body to transmit the photoacoustic signals; a communication quality acquiring unit configured to acquire a communication quality of wireless communication which the communication unit performs; a memory configured to store the photoacoustic signals; and a determining unit configured to determine, according to the communication quality, a communication rate of the communication unit and whether to store the photoacoustic signals in the memory.

### BACKGROUND OF THE INVENTION

### Field of the Invention

The present invention relates to a photoacoustic probe.

### Description of the Related Art

A photoacoustic tomography (PAT) is known as one of methods for obtaining optical characteristic values such as an absorption coefficient inside an object. An apparatus which uses PAT (hereinafter referred to as a photoacoustic apparatus) irradiates pulsed light generated from a light source to a living body. The photoacoustic apparatus receives, using a probe, acoustic waves (photoacoustic waves) generated when light having propagated through the object while diffusing into the object is absorbed by a light absorber. By analyzing this reception signals, an initial acoustic pressure distribution resulting from the light absorber inside the object is obtained. Expression (1) below indicates an acoustic pressure of photoacoustic waves obtained from the light absorber.

P=Γ·μₐ·Φ (1)

Here, P is an initial acoustic pressure. Γ is a Gruneisen coefficient which is an elastic characteristic value and which is a division of the product of a volume expansion coefficient β and the square of the speed of sound c by the specific heat capacity Cₚ. μₐ is an absorption coefficient of the light absorber, and Φ is a quantity of light absorbed by the light absorber.

As understood from Expression (1), an absorption coefficient can be obtained from an initial acoustic pressure at an arbitrary position and the quantity of light arriving at that position. Since an absorption coefficient is different depending on a light absorber, by obtaining a distribution of absorption coefficients of an object, a distribution of light absorbers that form the object (for example, a distribution of blood vessels or the like) is known. Moreover, by using a light source capable of radiating light having a plurality of wavelengths, it is possible to obtain an oxygen saturation and a substance concentration inside the object.

In a photoacoustic apparatus, the use of a handheld probe capable of accessing an observation segment easily similarly to an ultrasound diagnosis apparatus has been researched and developed. In an ultrasound diagnosis apparatus in which the use of a handheld probe progresses, a technique of performing information communication between an ultrasound probe and an apparatus body wirelessly has been developed. According to this wireless communication ultrasound diagnosis apparatus, inconveniences of processing resulting from the use of cables are eliminated.

Japanese Patent Application Publication No. 2009-066046 discloses a configuration in which a semiconductor memory is disposed inside an ultrasound probe head included in an ultrasound diagnosis apparatus so that the semiconductor memory is used for storing and managing ultrasound measurement data.

Japanese Patent Application Publication No. 2014-50648 discloses a configuration in which quality of wireless communication is monitored and the amount of data communication is reduced according to a quality index value in order to improve real-time properties of ultrasound images in an ultrasound apparatus in which an ultrasound probe and an apparatus body perform wireless communication.

### SUMMARY OF THE INVENTION

When a handheld photoacoustic apparatus wirelessly transmits photoacoustic signals based on photoacoustic waves received by a photoacoustic probe to an apparatus body, there is a problem that communication quality may decrease depending on an ambient environment and an apparatus state and it may be difficult to transmit data at a predetermined communication rate. Moreover, in a case where data is stored in a memory inside a photoacoustic probe, there is a problem that, if there is a small storable data capacity (that is, available capacity), it is not possible to store all the measured photoacoustic signals.

In view of the above problems, it is an object of the present invention to provide a technique of obtaining data satisfactorily as much using a photoacoustic probe that communicates with a photoacoustic apparatus body.

According to an aspect of the present invention, there is provided a photoacoustic probe including: a receiving unit configured to receive photoacoustic waves generated from an object irradiated with light; a signal processing unit configured to perform a process for outputting photoacoustic signals originating from the photoacoustic waves; a communication unit configured to perform wireless communication with a photoacoustic apparatus body to transmit the photoacoustic signals; a communication quality acquiring unit configured to acquire a communication quality of wireless communication which the communication unit performs; a memory configured to store the photoacoustic signals; and a determining unit configured to determine, according to the communication quality, a communication rate of the communication unit and whether to store the photoacoustic signals in the memory.

According to the present invention, it is possible to provide a technique of obtaining data satisfactorily as much using a photoacoustic probe that communicates with a photoacoustic apparatus body.

Further features of the present invention will become apparent from the following description of exemplary embodiments with reference to the attached drawings.

### BRIEF DESCRIPTION OF THE DRAWINGS

FIG. 1 is a block diagram of a photoacoustic apparatus according to a first embodiment;

FIGS. 2A and 2B are schematic diagrams of a handheld probe according to the first embodiment;

FIG. 3 is a block diagram illustrating a computer and a peripheral configuration according to the first embodiment;

FIGS. 4A and 4B are diagrams illustrating a process flow according to the first embodiment;

FIG. 5 is a diagram illustrating the contents set by a determining unit according to the first embodiment; and

FIGS. 6A to 6D are timing charts of a system operation according to a second embodiment.

### DESCRIPTION OF THE EMBODIMENTS

Hereinafter, preferred embodiments of the present invention will be described with reference to the drawings. Dimensions, materials, shapes, relative arrangements, and the like of constituent components described below are to be appropriately changed according to the configuration and various conditions of an apparatus to which the present invention is applied. Therefore, the scope of the present invention is not limited to those described below.

The present invention relates to a technique of detecting photoacoustic waves propagating from an object, storing photoacoustic signals which are signals based on the detected photoacoustic waves or photoacoustic data based on the photoacoustic signals, and generating characteristic information (object information) inside the object from the photoacoustic signals or the photoacoustic data. The present invention also relates to a method for generating and displaying images indicating characteristic information inside an object. Therefore, the present invention can be understood as a photoacoustic probe and a photoacoustic apparatus, and a method for controlling the same. The present invention can be also understood as an acquisition method, a storing method, a communication method, and a signal processing method for photoacoustic signals and photoacoustic data based thereon. The present invention can be also understood as a program for causing an information processing device including hardware resources such as a CPU and a memory to execute these methods and a computer-readable non-transitory storage medium having the program stored therein.

The photoacoustic probe and the photoacoustic apparatus according to the present invention include a photoacoustic imaging apparatus which uses a photoacoustic effect to irradiate light (electromagnetic waves) to an object to receive acoustic waves generated inside the object and acquire characteristic information of the object as image data. The characteristic information in the photoacoustic apparatus is information on a characteristic value corresponding to each of a plurality of positions inside the object, generated using signals originating from the received photoacoustic waves. The photoacoustic image data according to the present invention is a concept including all kinds of image data originating from photoacoustic waves generated by radiation of light, photoacoustic signals converted from the photoacoustic waves, and photoacoustic data obtained by applying various signal processes such as addition or correction to the photoacoustic signals.

For example, the photoacoustic image data is image data indicating a spatial distribution of at least one piece of object information such as an acoustic pressure of photoacoustic waves (an initial acoustic pressure), an absorption energy density, and an absorption coefficient. Photoacoustic image data indicating spectral information such as concentrations of substances constituting an object is obtained on the basis of photoacoustic waves generated by radiation of light having a plurality of different wavelengths. The photoacoustic image data indicating the spectral information may be an oxygen saturation, a value obtained by weighting the oxygen saturation by an intensity such as an absorption coefficient, a total hemoglobin concentration, an oxyhemoglobin concentration, or a deoxyhemoglobin concentration. Moreover, the photoacoustic image data indicating the spectral information may be a glucose concentration, a collagen concentration, a melanin concentration, or a volume fraction of fats or water.

A two-dimensional or three-dimensional characteristic information distribution is obtained on the basis of the characteristic information of each position inside an object. The distribution data may be generated as image data. The characteristic information may be obtained as distribution information at respective positions inside the object rather than numerical data. That is, the characteristic information is distribution information such as an initial acoustic pressure distribution, an energy absorption density distribution, an absorption coefficient distribution, or an oxygen saturation distribution. The distribution data is also referred to as photoacoustic image data or reconstructed image data.

Acoustic waves referred in the present invention are typically ultrasound waves and include elastic waves called sound waves or acoustic waves. Electrical signals converted from acoustic waves by a transducer or the like are also referred to as acoustic signals. However, the expressions of ultrasound waves or acoustic waves referred in the present specification do not restrict the wavelength of those elastic waves. The acoustic waves generated by the photoacoustic effect are referred to as photoacoustic waves or light-induced ultrasound waves. Electrical signals originating from photoacoustic waves are also referred to as photoacoustic signals. The “photoacoustic signals” in the present specification may include signals converted from photoacoustic waves and data obtained by applying various processes to the signals.

In the following description, a photoacoustic apparatus that irradiates pulsed light to an object, receives photoacoustic waves from the object, and generates a blood vessel image (a structure image) inside the object is employed as a object information acquisition apparatus. In the following embodiments, a photoacoustic apparatus in which a photoacoustic apparatus body and a photoacoustic probe exchange information by wireless communication of electric waves is employed. However, the present invention can be applied to a photoacoustic apparatus in which information is exchanged, for example, by optical communication, electrical wires, or optical fiber instead of wireless communication.

### First Embodiment

### Overall Configuration

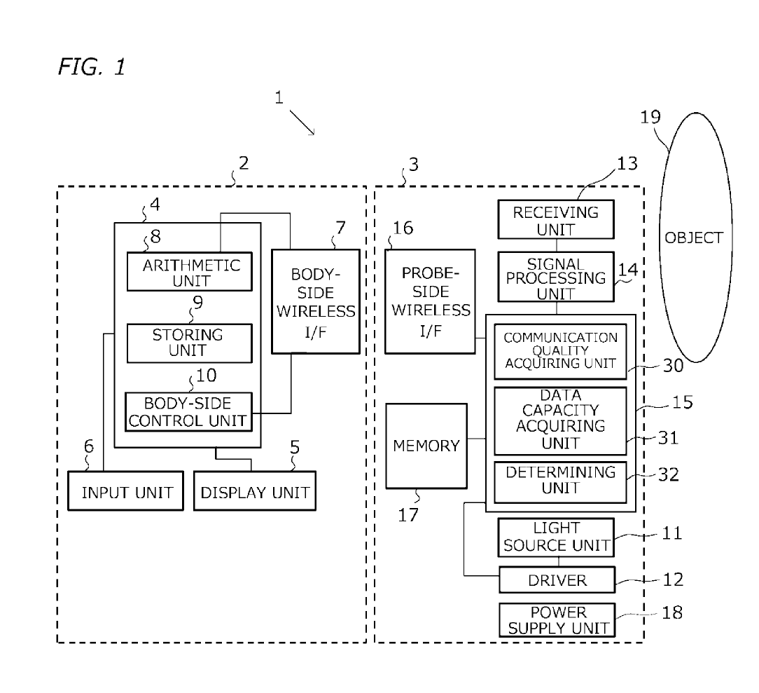

A configuration of a photoacoustic apparatus 1 according to the present embodiment will be described with reference to a block diagram of FIG. 1. The photoacoustic apparatus 1 includes a photoacoustic apparatus body 2 and a probe 3. The photoacoustic apparatus body 2 includes a computer 4, a display unit 5, an input unit 6, a body-side wireless interface 7 (a body-side wireless I/F). The computer 4 includes an arithmetic unit 8, a storing unit 9, a body-side control unit 10. The probe 3 includes a light source unit 11, a driver 12, a receiving unit 13, a signal processing unit 14, a probe control unit 15, a probe-side wireless interface 16 (a probe-side wireless I/F), a memory 17, a power supply unit 18.

The driver 12 drives the light source unit 11 in a first cycle (an emission cycle) under the control of the probe control unit 15 and irradiates pulsed light toward an object 19. In this way, photoacoustic waves is generated from the object 19 in the first cycle (the emission cycle). The receiving unit 13 receives photoacoustic waves generated from the object 19 in the first cycle (the emission cycle) and outputs electrical signals (photoacoustic signals) as analog signals. That is, the receiving unit 13 receives photoacoustic waves at intervals defined by the first cycle (the emission cycle).

The signal processing unit 14 performs a process of outputting photoacoustic signals originating from photoacoustic waves. For example, the signal processing unit 14 converts the analog photoacoustic signals output from the receiving unit 13 to digital signals, performs a correction process and an amplification process, and then outputs the processed signals. The probe control unit 15 has the function of a memory controller and stores the digital photoacoustic signals in the memory 17. Moreover, the probe control unit 15 outputs the photoacoustic data to the computer 4 via the probe-side wireless interface 16 and the body-side wireless interface 7.

The computer 4 combines the photoacoustic data output from the wireless interface 7 every first cycle (emission cycle) using the arithmetic unit 8, the storing unit 9, and the body-side control unit 10 according to a second cycle (hereinafter also referred to as a cycle of an imaging frame rate). The combined data based on the photoacoustic signal is stored in the storing unit 9. Here, combination is not limited to simple addition but includes weighted addition, averaging, moving averaging, and the like. Although averaging will be described as an example, a combination method other than the averaging may be applied. The computer 4 performs a process such as image reconstruction with respect to the data based on the photoacoustic signals stored in the storing unit 9 to generate photoacoustic image data within a period defined by the second cycle (the cycle of the imaging frame rate).

The display unit 5 displays images on the basis of the photoacoustic image data of the second cycle (the cycle of the imaging frame rate). In a case where the display unit 5 could not display the photoacoustic image data at the second cycle (the cycle of the imaging frame rate), the photoacoustic image data may be converted to a frame rate appropriate for display on the display unit 5 using a frame rate converter. The computer 4 may perform imaging processing for display or a process of combining graphics for GUI with respect to the obtained photoacoustic image data as necessary.

In the present specification, the embodiments are described using the terms of the first cycle (the emission cycle) and the second cycle (the cycle of the imaging frame rate). However, the “cycle” herein does not need to be an “absolutely constant repeating period”. That is, in the present specification, the terminal “cycle” is used even in a case where an event occurs repeatedly at non-constant time intervals. In a case where a pause period is included in the first cycle, particularly, a repetition period in a period which does not include a pause period may be referred to as a “cycle”.

A user (a physician, an engineer, or the like) observes photoacoustic images displayed on the display unit 5. The images displayed on the display unit 5 may be stored in a memory in the computer 4, a data management system connected to the photoacoustic apparatus via a communication network, or the like on the basis of a storage instruction from the user or the computer 4. The display unit 5 may be included in the photoacoustic apparatus body 2 and may be provided separately from the photoacoustic apparatus body 2.

The input unit 6 receives an instruction input from the user. The body-side control unit 10 delivers the instruction from the user and setting values from the photoacoustic apparatus body to the probe control unit 15 in the probe 3 via the wireless interface. The setting values include, for example, the first cycle (emission cycle), the number of repetitions, an intensity of a light source, an A/D conversion rate (the frequency of converting analog signals to digital signals), and the like. The probe control unit 15 performs control using transmitted information.

Detailed Configuration of Each Block

Next, a preferred configuration of each block will be described in detail.

Probe 3

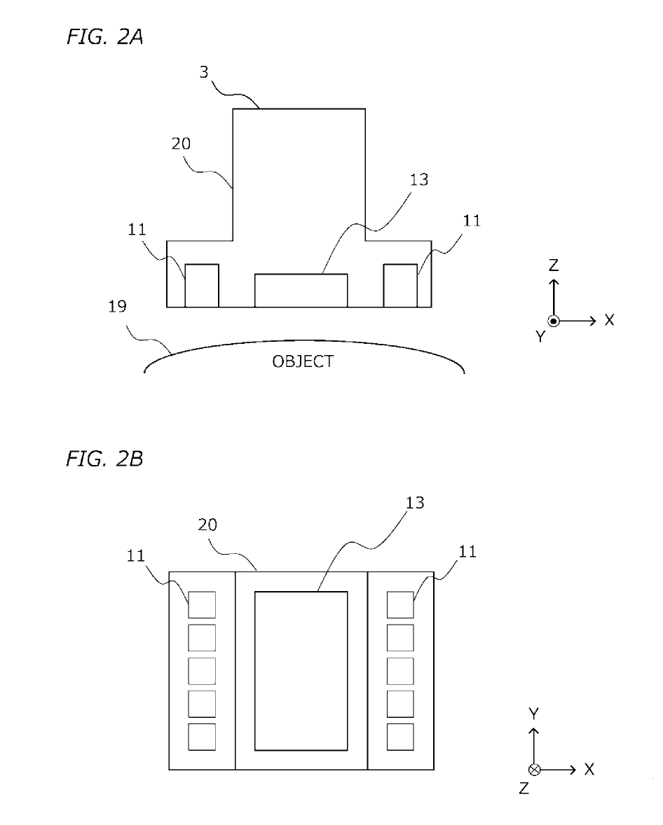

FIG. 2A is a schematic cross-sectional view illustrating a main configuration of the probe 3 according to the present embodiment. The probe 3 has a configuration in which the light source unit 11, the driver 12, the receiving unit 13, the signal processing unit 14, the probe control unit 15, the wireless interface 16, the memory 17, and the power supply unit 18 are surrounded by a housing 20 which is a casing. Components other than the light source unit 11 and the receiving unit 13 are not illustrated for better understanding of the drawing. The user may use the probe 3 as a handheld probe by grasping the housing 20.

Light Source Unit 11

The light source unit 11 generates light for radiation to the object 19. It is preferable to use a semiconductor light emitting device such as a semiconductor laser or a light emitting diode from the need of mounting the light source unit 11 into the housing 20 of the probe 3. However, a light source such as the other lasers and a flash lamp can be also used. In a case where it is desired to acquire an oxygen saturation and a substance concentration, a plurality of types of semiconductor lasers or light emitting diodes that generate light of different wavelengths may be used. A pulse width of light generated by the light source unit 11 is at least 10 ns and not more than 1 μs, for example.

Although a wavelength of at least 400 nm and not more than 1600 nm is suitably used as the wavelength of light, the wavelength may be determined depending on light absorption characteristics of a light absorber, which are to be imaged. In a case where blood vessels are imaged with a high resolution, light of a wavelength (at least 400 nm and not more than 800 nm) in which a large amount of light is absorbed in blood vessels may be used. In a case where a deep part of a living body is imaged, light of a wavelength (at least 700 nm and not more than 1100 nm) in which a small amount of light is absorbed in background tissues (water, fats, and the like) of a living body may be used. In a case where a semiconductor light emitting device is used as the light source unit 11, photoacoustic signals obtained by one radiation may not have a desired S/N ratio due to a deficient light quantity. In this case, the photoacoustic signals output at the first cycle (the emission cycle) may be averaged by the signal processing unit 14 to improve the S/N ratio, and the photoacoustic image data is calculated at the second cycle (the cycle of the imaging frame rate) on the basis of the averaged photoacoustic signal.

In the present embodiment, a first wavelength of a radiation light is 797 nm. Since light of this wavelength reaches a deep part of an object and the absorption coefficients of oxyhemoglobin and deoxyhemoglobin are approximately the same, the light of this wavelength is suitable for detecting a blood vessel structure. Moreover, when an oxygen saturation is calculated, a second wavelength is 756 nm.

FIG. 2B is an example of an arrangement of the light source unit 11 and the receiving unit 13 at a distal end of the housing 20. On a reception surface, light emitting ends of the plurality of semiconductor light emitting devices included in the light source unit 11 are arranged around the receiving unit 13. According to this configuration, light can be irradiated to a wide region of an object. Moreover, the wavelengths of a radiation light may be changed for respective light emitting ends.

Driver 12

The driver 12 drives the light source unit 11 at the first cycle (the emission cycle) according to an instruction of the probe control unit 15 to cause the light source unit 11 to emit light. In a case where a plurality of light emitting diodes or semiconductor lasers is used as the light source unit 11, a large optical power is necessary for obtaining photoacoustic signals from an object. Therefore, it is necessary to supply a large current to the driver 12. Due to this, it is preferable to design wirings of the driver 12 and the light source unit 11 so as not to have inductance components as much as possible.

Receiving Unit 13

The receiving unit 13 includes a transducer that receives photoacoustic waves generated with emission of light at the first cycle (the emission cycle) and outputs electrical signals and a support that supports the transducer. Transducers formed of piezoelectric materials, capacitive micro-machines ultrasonic transducers (CMUT), transducers which use Fabry-Perot interferometers can be used as the transducer. Examples of piezoelectric materials include a piezoelectric ceramic material such as PZT (lead zirconate titanate) and a polymer piezoelectric film material such as PVDF (polyvinylidene fluoride).

Electrical signals obtained by the transducer every first cycle (emission cycle) are time-resolved signals. Due to this, an amplitude of electrical signals indicates a value (a value proportional to an acoustic pressure) based on the acoustic pressure received by the transducer at respective time points. A transducer capable of detecting a frequency component (typically from 100 KHz to 10 MHz) that form photoacoustic waves is preferably used as the transducer. Moreover, it is also preferable that a plurality of transducers are arranged on the support to form a flat surface or a curved surface called 1D array, 1.5D array, 1.75D array, or 2D array. Furthermore, in order to detect acoustic waves from various angles to improve image accuracy, it is preferable to arrange transducer such that the transducers surround the entire circumference of the object 19. Moreover, in a case where the object 19 is such large that the entire circumference is not surrounded by the transducers, the transducers may be arranged on a hemispherical support. However, in a case where a handheld photoacoustic probe is used, the handleability of a photoacoustic probe may not be impaired.

A medium that propagates photoacoustic waves may be disposed in a space between the receiving unit 13 and the object 19. In this way, acoustic impedance matching is realized at the interface between the object 19 and the transducer. Examples of the medium include water, oil, and ultrasound gel.

The photoacoustic apparatus 1 may include a holding member that holds the object 19 to stabilize the shape. A member having high light transmitting properties and high acoustic wave transmitting properties is preferably used as the holding member. For example, polymethyl pentene, polyethylene terephthalate, acryl, and the like can be used.

In a case where the apparatus according to the present embodiment generates ultrasound images by transmitting and receiving acoustic waves as well as generating photoacoustic images, the transducer may function as transmitting means for transmitting acoustic waves. A transducer as receiving means and a transducer as transmitting means may be a single (common) transducer and may be different transducers.

Signal Processing Unit 14

The signal processing unit 14 includes an amplifier that amplifies electrical signals which are analog signals output from the receiving unit 13, generated with emission of light every first cycle (emission cycle) and an A/D converter that converts the analog signals output from the amplifier to digital signals. The amplifier may be configured such that an amplification factor is variable, and the signal processing unit 14 may be formed of a field programmable gate array (FPGA).

In a case where the receiving unit 13 includes a plurality of transducers arranged in an array form, the analog signals output by the respective transducers are amplified by a plurality of corresponding amplifiers and are converted to digital signals by a plurality of corresponding A/D converters. An A/D conversion rate is preferably equal to or larger than at least twice the bandwidth of input signals. Therefore, in a case where the frequency component of photoacoustic waves is from 100 KHz to 10 MHz, the A/D conversion rate is 20 MHz or higher, and preferably, 40 MHz.

The signal processing unit 140 synchronizes the timing of radiation of light with the timing of a signal collection process using an emission control signal. That is, the signal processing unit 140 starts A/D conversion at a predetermined A/D conversion rate according to emission time points. As a result, every first cycle (emission cycle), it is possible to acquire a digital data stream originating from photoacoustic waves at a timing interval (an A/D conversion interval) corresponding to 1/(A/D conversion rate) from the emission time point. The signal processing unit 140 is also referred to as a data acquisition system (DAS).

Probe Control Unit 15

The probe control unit 15 controls the emission timings of the light source unit 11, the A/D conversion rate, and timings and acquires photoacoustic signals every emission of the light source unit 11. The probe control unit 15 transmits the photoacoustic data to the computer 4 via the probe-side wireless interface 16 and the body-side wireless interface 7. Moreover, the probe control unit 15 has the function of a memory controller and writes photoacoustic signals acquired every emission of the light source unit 11 in the memory 17. The probe control unit 15 can be configured as a processor, a processing circuit, or the like. Each block inside the probe control unit 15 may be configured as a processing circuit having a physical entity and may be realized as a functional block by means of a program module or the like. In a case where the probe is not a handheld probe but a mechanically scanning probe which uses a scanning mechanism such as an XY stage, the probe control unit 15 moves the probe along a predetermined path.

A communication quality acquiring unit 30 acquires the quality of wireless communication between the apparatus body and the photoacoustic probe. The communication quality changes according to an ambient environment (for example, noise from surrounding electronic apparatuses, electromagnetic interference, weather when used outside, and the like) and an apparatus state (an aging state of components of the probe and the apparatus body, the distance between the probe and the apparatus, and the like). Therefore, the communication quality acquiring unit 30 measures an execution speed and a data delay amount during communication according to a method to be described later to determine the communication quality.

A data capacity acquiring unit 31 performs communication with the memory 17 to acquire an available capacity (a data capacity storable in the memory 17).

The determining unit 32 determines a communication rate between the apparatus body and the photoacoustic probe and the storage rate in the memory 17 on the basis of the results obtained by the communication quality acquiring unit 30 and the data capacity acquiring unit 31, and sets the rates to the apparatus and the probe. The storage rate in the memory 17 includes a case in which a stored data amount is zero (that is, data is not stored). In other words, the determining unit 32 can also determine whether to store photoacoustic signals in the memory 17.

Wireless Interfaces 7 and 16

The body-side wireless interface 7 and the probe-side wireless interface 16 perform bidirectional communication. For example, a wireless interface conforming to the wireless LAN standard such as Wi-Fi is suitable. In a case where light is irradiated to an object at a first cycle, at least a communication speed with which photoacoustic signals originating from photoacoustic waves obtained by one radiation of light can be transmitted to the photoacoustic apparatus body until the next radiation of light is calculated. Moreover, the setting data and the like from the photoacoustic apparatus body are also transmitted to the probe 3 via the wireless interface. The photoacoustic signals which are transmission data transmitted from the probe-side wireless interface 16 may be the raw data itself output from the signal processing unit 14 and may be data obtained through various signal processes such as addition, correction, or thinning-out. The probe-side wireless interface 16 corresponds to a communication unit included in the photoacoustic probe of the present invention.

Memory 17

The memory 17 is configured to store photoacoustic signals temporarily or permanently, and a detachable nonvolatile memory (typically, a flash memory such as a memory card or a USB memory) is suitably used. In a case where a nonvolatile memory is used, the memory 17 may be detached from the probe 3 after measuring photoacoustic signals and the photoacoustic signals may be read into the photoacoustic apparatus body or another computer. Moreover, after a memory (a flash memory) in which photoacoustic signals are written is detached from the probe 3, by attaching another memory having a sufficient data capacity for writing data to the probe 3, reading of data and measurement of photoacoustic signals may be performed in parallel. In a case where a USB memory or a memory card which is highly versatile is used as a flash memory, it is not necessary to prepare a special reader in a case where the photoacoustic apparatus body or the like reads photoacoustic signals. The memory 17 corresponds to a memory of the present invention.

In a case where a volatile memory such as RAM is used as the memory 17, power is always applied to the memory 17 so that the stored photoacoustic signals do not disappear. After photoacoustic signals are measured, the photoacoustic signals are transmitted to the photoacoustic apparatus body or the like via a wireless interface or a priority interface (not illustrated). In a case where data stored in the memory 17 is transmitted, the probe control unit 15 may erase the data having been transmitted from the memory 17. In this way, it is possible to increase a writable storage area of the memory 17 to secure a data capacity.

Power Supply Unit 18

The power supply unit 18 is mounted inside the housing 20 to supply an electric power to respective blocks in the probe 3. Since a large electric power is required for a light source to emit light, secondary batteries such as a nickel-metal hydride battery, a lithium ion battery, or a lithium polymer battery having a high energy density are suitably used as the power supply unit 18. Moreover, by using rechargeable batteries, it is possible to eliminate the time and labor incurred in replacement of batteries. Rather than using secondary batteries, the photoacoustic apparatus body or the power supply unit and the photoacoustic probe may be connected by a power cable (not illustrated) to supply an electric power thereto. In this case, due to presence of the power cable, although the degree of freedom of operating the photoacoustic probe decreases, it is possible to prevent interruption of measurement due to discharge of the battery. Moreover, a power cable and a secondary battery may be used together.

The photoacoustic probe may include a notifying unit (not illustrated) that notifies the user of at least one of a present communication quality and an available capacity of the memory 17. Particularly, a notification may be issued in a case where a communication quality deteriorates or an available capacity is small. Arbitrary means such as an audio speaker or an alarming LED may be used for notification.

Computer 4

The computer 4 includes an arithmetic unit 8, a storing unit 9, and a body-side control unit 10. A unit that performs an arithmetic function of the arithmetic unit 8 may be configured as a processor such as a CPU or a graphics processing unit (GPU) and an arithmetic circuit such as a field programmable gate array (FPGA). These units may be configured as a single processor or a single arithmetic circuit and may be configured as a plurality of processors or arithmetic circuits.

The arithmetic unit 8 of the computer 4 sums and averages pieces of data obtained at the same time point from the emission time points among a digital data array output from the wireless interface 7 every first cycle (emission cycle). The averaged digital signals are stored in the storing unit 9 as averaged photoacoustic signals every second cycle (cycle of the imaging frame rate).

The arithmetic unit 8 generates reconstructed photoacoustic image data (structure images or functional images) and executes other arithmetic processes on the basis of the averaged photoacoustic signals stored in the storing unit 9 every second cycle (cycle of the imaging frame rate). The arithmetic unit 8 may receive various parameters such as measurement conditions, an acoustic velocity of the object, and a configuration of a holding portion from the input unit 6 and may use the parameters in arithmetic processes.

An arbitrary method such as back-projection on a time domain, back-projection on a Fourier domain, or a model-base method (a repetition computation method) may be used as a reconstruction algorithm used for the arithmetic unit 8 to generate photoacoustic image data. Examples of the back-projection on the time domain include universal back-projection (UBP), filtered back-projection (FBP), and delay-and-sum.

In a case where the light source unit 11 can switchably emit light of two wavelengths, the arithmetic unit 8 may generate a first initial acoustic pressure distribution from photoacoustic signals originating from light of the first wavelength and a second initial acoustic pressure distribution from photoacoustic signals originating from light of the second wavelength by an image reconstruction process. The arithmetic unit 8 acquires a first absorption coefficient distribution by correcting the first initial acoustic pressure distribution using a light intensity distribution of the light of the first wavelength and acquires a second absorption coefficient distribution by correcting the second initial acoustic pressure distribution using a light intensity distribution of the light of the second wavelength. The arithmetic unit 8 acquires an oxygen saturation distribution from the first and second absorption coefficient distributions. The content and the order of computations for obtaining the oxygen saturation distribution are not limited thereto.

The storing unit 9 is configured as a volatile memory such as a random access memory (RAM) or a non-transitory storage medium such as a read only memory (ROM), a magnetic disk, or a flash memory. The storage medium in which a program is stored is a non-transitory storage medium. The storing unit 9 may be configured as a plurality of storage media. The storing unit 9 can store various pieces of data such as photoacoustic data averaged at the second cycle (the cycle of the imaging frame rate), photoacoustic image data generated by the arithmetic unit 8, and reconstructed image data based on the photoacoustic image data.

The body-side control unit 10 is configured as an arithmetic device such as CPU. The body-side control unit 10 controls operations of respective components of the photoacoustic apparatus. When controlling the respective components, the body-side control unit 10 controls the operations on the basis of an instruction signal (for example, a measurement start instruction) input from the input unit 6, a program code stored in the storing unit 9, and the like. The body-side control unit 10 transmits an instruction from the user and the setting values (the first cycle (emission cycle), the number of repetitions, an intensity of a light source, and an A/D conversion rate) from the photoacoustic apparatus body to the probe control unit 15 via the wireless interface.

The computer 4 may be a workstation designed for a dedicated use and may be a general-purpose PC or workstation which operates according to an instruction of a program stored in the storing unit 9. The respective components of the computer 4 may be configured as different hardware components. At least some components of the computer 4 may be configured as the same hardware.

The computer 4 may notify the user of at least one of the present communication quality and the available capacity of the memory 17. Particularly, a notification may be issued in a case where a communication quality deteriorates or an available capacity is small. Arbitrary means such as an audio speaker or an alarming LED other than the display unit 5 may be used for notification.

Display Unit 5

The display unit 5 is configured as a display device such as a liquid crystal display or an organic EL display and displays images, numerical values of specific positions, and the like based on the object information or the like obtained by the computer 4. The display unit 5 displays reconstructed image data of a frame rate (imaging frame rate) of the second cycle. Generally, a frame rate of 50 Hz, 60 Hz, 72 Hz, or 120 Hz is used. In a case where the frame rate (the imaging frame rate) of the second cycle is matched to the frame rate of the display unit 5, a frame rate conversion unit 26 to be described later may be eliminated. The display unit 5 may display a GUI for operating images and the apparatus. Before displaying images on the display unit 5, image processing such as adjustment of a brightness value of the computer 4 may be performed. The display unit 5 may display a charge state of the power supply unit 18 of the probe 3 and a use state of the memory 17.

Input Unit 6

The input unit 6 is a device which is operated by a user to input an instruction, and an operation console or the like including a mouse, a keyboard, and a dedicated knob can be employed. A touch panel which serves as the display unit 5 and the input unit 6 may be used. The input unit 6 receives instructions, numerical values, and the like input from the user and delivers the same to the computer 4. The photoacoustic apparatus body also includes an interface for reading the content of the memory 17 (for example, an interface conforming to USB standards or IEEE 1394 standards, a memory card reader, and the like). The user can input instruction for starting and ending measurement and an instruction for storing created images using the input unit 6.

The respective components of the photoacoustic apparatus may be configured as different devices and may be configured as an integrated device. At least some components of the photoacoustic apparatus may be configured as an integrated device.

Configuration Example of Computer

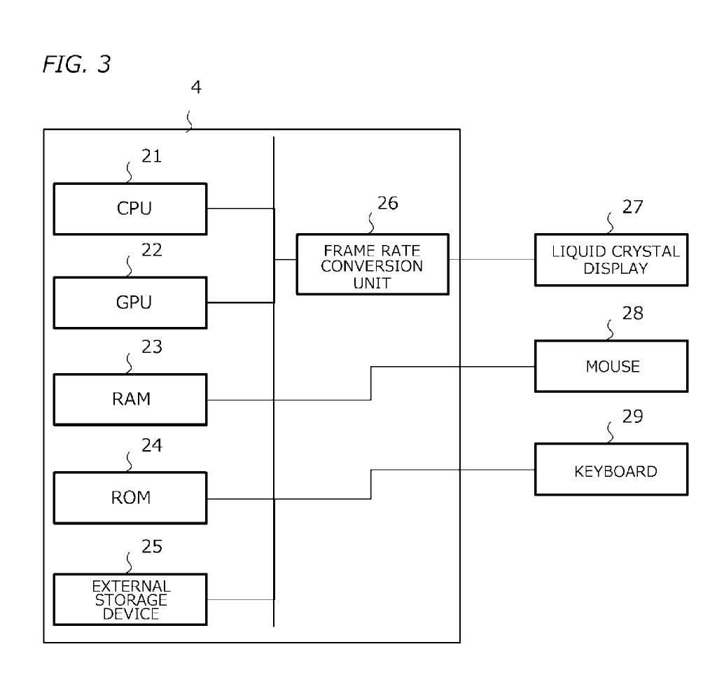

FIG. 3 illustrates a specific configuration example of the computer 4 according to the present embodiment. The computer 4 according to the present embodiment includes a CPU 21, a GPU 22, a RAM 23, a ROM 24, and an external storage device 25. The frame rate conversion unit 26 converts photoacoustic image data to a frame rate appropriate for display on the liquid crystal display 27. Moreover, the computer 4 is connected to a liquid crystal display 27 as the display unit 5 and a mouse 28 and a keyboard 29 as the input unit 6. Furthermore, the computer 4 may be realized by cooperation of a plurality of information processing devices. An information processing device provided at a remote site and provided by a cloud computing service or the like may be used as the computer 4.

Object 19

The object 19 will be described although the object does not constitute the photoacoustic apparatus. The photoacoustic apparatus according to the present embodiment can be used for examining malignant tumors, blood vessel diseases, and the like of a person or an animal and observing the progress of chemical treatments. Therefore, the object 19 may be a living body, and specifically, the breast, the organs, the vascular plexus, the head, the neck, the abdomen, the limbs including the fingers and the toes, and the like of a human body or an animal may be an examination target segment. For example, in a case where a human body is a measurement target, oxyhemoglobin or deoxyhemoglobin, a blood vessel that contains many oxyhemoglobins or deoxyhemoglobins, or new-born blood vessels formed near a tumor may be the target of a light absorber. Moreover, plaque on the carotid wall or the like may be the target of a light absorber. Furthermore, dyes such as methylene blue (MB) or indocyanine green (ICG), fine gold particles, and substances introduced from the outside and obtained by modifying these materials in an integrated or chemical manner may be a light absorber. Furthermore, a puncture needle or a light absorber attached to the puncture needle may be an observation target. The object may be a nonliving object such as a phantom or a testing object.

Process Flow

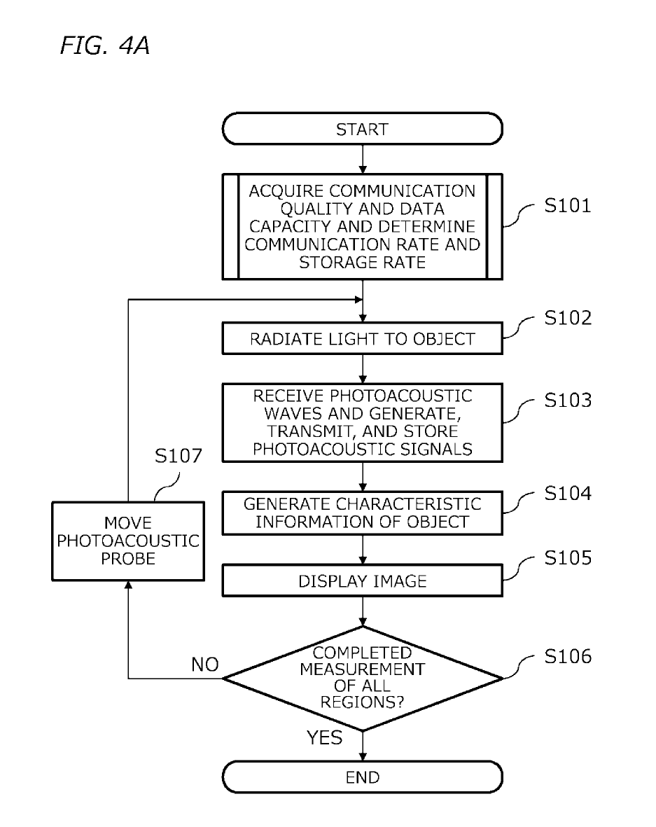

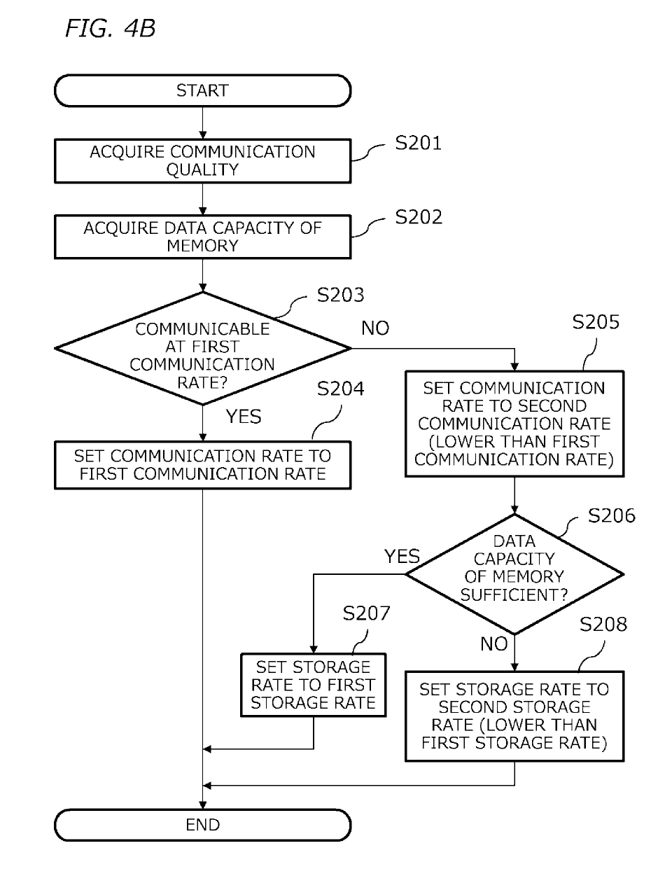

Referring to FIG. 4A, an overall process flow of the present embodiment will be described. In step S101, the probe control unit 15 acquires the communication quality and the data capacity of a memory and determines a communication rate and a storage rate of data (photoacoustic signals). This process will be described in detail later. In step S102, the light source unit 11 irradiates light to an object under the control of the driver 12. In step S103, the receiving unit 13 receives photoacoustic waves generated from the object to output photoacoustic signals and the signal processing unit 14 converts the photoacoustic signals to digital signals. The probe control unit 15 controls transmission of photoacoustic signals using the probe-side wireless interface 16 and storage of the photoacoustic signals in the memory 17 according to the content determined in S101.

In step S104, the computer 4 reconstructs the photoacoustic signals received via the body-side wireless interface 7 to generate photoacoustic image data. In step S105, the display unit 5 displays the photoacoustic images inside the object at a predetermined imaging frame rate.

In step S106, the computer 4 determines whether measurement of all regions has ended. In a case where the photoacoustic probe is a mechanically scanning probe, measurement ends at a time point at which acquisition of data from a measurement region that a user has set using an input unit is completed. In a case where the photoacoustic probe is a handheld probe, measurement ends in a case where a stop instruction (for example, pressing of a switch) input by a user is detected or in a case where movement of the probe away from an object is detected. On the other hand, in a case where measurement has not ended, the flow proceeds to step S107 and the photoacoustic probe is moved by a scanning mechanism or by the hands of the user.

FIG. 4B is a flowchart for describing step S101 in detail. In step S201, the communication quality acquiring unit 30 acquires the quality of wireless communication between the photoacoustic probe and the apparatus body.

In this example, a communication speed of data is used as the communication quality. The communication quality acquiring unit 30 measures the amount of data that the body-side wireless interface 7 has received from the probe-side wireless interface 16 within a predetermined unit period to calculate a communication speed and compares the calculated value with a threshold to acquire a communication quality. The communication quality may be represented in two steps of good and bad and may be represented in multiple steps according to the number of thresholds. Alternatively, the communication quality acquiring unit 30 may convert the value of the communication speed using a table or a numerical equation stored in advance in a memory to calculate the communication quality. Preferably, the communication quality acquiring unit 30 measures the communication speed a plurality of times to calculate an average value.

The photoacoustic apparatus can preferably operate in a preparation mode for acquiring a communication quality and a data capacity to determine a communication rate and a storage rate in addition to a mode for measuring an object actually. Moreover, it is preferable that test data for use in the preparation mode is stored in the memory 17. The test data is communication data in which data having a predetermined size is inserted in a header portion, for example. The communication quality acquiring unit 30 measures a communication speed by transmitting and receiving the test data. Moreover, a plurality of pieces of test data having different sizes may be prepared and be selectively used depending on measurement conditions and the like.

A delay until data transmitted from the probe-side wireless interface 16 reaches the body-side wireless interface and an intensity of wireless signals transmitted and received between the probe-side wireless interface and the body-side wireless interface may be used as the communication quality. Moreover, data reproducibility or an error rate obtained by comparing data transmitted from the probe with data received by the apparatus body may be used as the communication quality.

In step S202, the data capacity acquiring unit 31 acquires a data capacity (an available capacity) storable in the memory 17.

In step S203, the determining unit 32 determines whether communication can be performed at a first communication rate on the basis of the acquired communication quality. Here, the first communication rate is a rate at which photoacoustic images can be displayed on the display unit 5 with high quality and a high frame rate. Typically, the first communication rate is a communication rate at which raw data of the photoacoustic signals can be transmitted to the apparatus body. In a case where a determination result of YES is obtained, the flow proceeds to step S204 and the determining unit 32 sets the communication rate to the first communication rate.

In a case where communication is performed at the first communication rate, photoacoustic signals may be stored in the memory 17 for the purpose of backup or the like. In this case, the determining unit 32 determines whether to store photoacoustic signals, as a stage prior to determining the storage rate. The fact that the photoacoustic signals are not stored can be considered that the storage rate is zero.

On the other hand, in a case where the communication quality is not good, the flow proceeds to step S205. The determining unit 32 sets the communication rate to a second communication rate lower than the first communication rate. Since the amount of data transmitted to the apparatus body is small due to the second communication rate, at least one of the image quality and the frame rate of the photoacoustic images displayed on the display unit 5 decreases.

The second communication rate may be a fixed value and may be changed according to a communication quality. For example, the determining unit 32 may set the second communication rate so that as large an amount of data as possible is transmitted within an allowable range of the communication quality.

In step S206, the determining unit 32 determines whether the data capacity of the memory 17 acquired in S202 can store the photoacoustic signals at the first storage rate. Here, the term “storage rate” indicates the percentage of the photoacoustic signals to be stored as compared to a case in which all acquired photoacoustic signals are stored. Typically, the first storage rate is a rate at which all pieces of raw data of the photoacoustic signals can be stored. The fact that “the storage rate is low” means that data is thinned out so that an amount of data smaller than the first storage rate is stored. In this step, first, the data capacity acquiring unit 31 calculates a total amount of data of the photoacoustic signals generated by photoacoustic measurement on the basis of the conditions (an image quality, a frame rate, an area of a measurement region, and the like) input using the input unit 6. The calculated total amount of data is compared with the data capacity acquired in S202.

In a case where a determination result of YES is obtained, the flow proceeds to step S207 and the determining unit 32 sets the storage rate to the first storage rate. The first storage rate herein is a rate at which all photoacoustic signals obtained according to the input conditions can be stored.

On the other hand, in a case where the calculated total amount of data is larger than an available capacity and the capacity is not sufficient, the determining unit 32 sets the storage rate to a second storage rate lower than the first storage rate in step S208. The second storage rate may be a fixed value and may be changed according to a data capacity. For example, the determining unit 32 sets the second storage rate so that as large an amount of data as possible within the range of an available capacity is stored. In this way, it is possible to prevent an image quality and a frame rate from decreasing too much.

Description of Operation

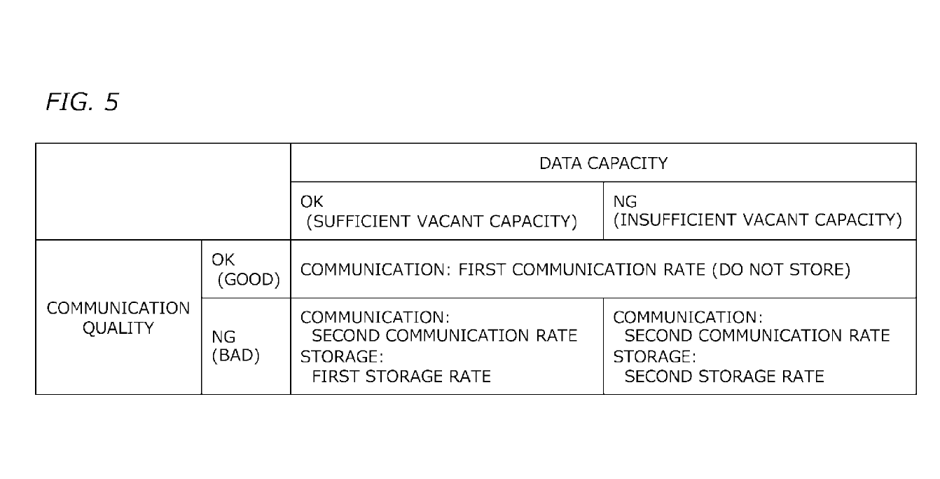

FIG. 5 is a table illustrating a specific setting example of a communication rate and a storage rate according to the above-described flow. In this example, the communication rate and the storage rate are selected from two steps. In a case where the communication speed is equal to or higher than a predetermined threshold, the communication quality is “good”. In this case, the photoacoustic probe irradiates light at the first cycle (emission cycle) to acquire photoacoustic signals and transmits the photoacoustic signals to the apparatus body via the wireless interface 16 at the first communication rate. The first communication rate herein indicates a rate at which all photoacoustic signals can be transmitted without thinning-out. Therefore, it is not necessary to store data in a memory regardless of an available capacity.

In a case where the communication speed is lower than the threshold, the communication quality is “bad”. In this case, the communication rate is decreased to the second communication rate. In this example, the second communication rate is a fixed value. In a case where images are displayed on the basis of the photoacoustic signals transmitted to the apparatus body at the second communication rate, it is preferable that such an image quality that images are useful for the user to manually operate the photoacoustic probe is obtained. In a case where the available capacity of a memory is sufficient, all photoacoustic signals are stored in the memory 17 without thinning-out. The stored data may be transmitted to the photoacoustic apparatus body 2 after surrounding wireless environment is improved and may be moved to the apparatus body by the user removing a card manually.

On the other hand, in a case where the communication quality is “bad” and the available capacity of a memory is not sufficient, the second communication rate is used for transmitting data and the second storage rate is used for storing data. In this way, even in a case where the remaining capacity of the memory is small, photoacoustic signals corresponding to one case can be stored reliably.

According to the present embodiment, in a case where data is acquired using a photoacoustic probe that performs communication with a photoacoustic apparatus body, photoacoustic signals can be transmitted and stored within an allowable range of a communication quality and an available memory capacity. As a result, photoacoustic images can be displayed with as satisfactory image quality as possible and as many photoacoustic signals as possible can be stored.

### Second Embodiment

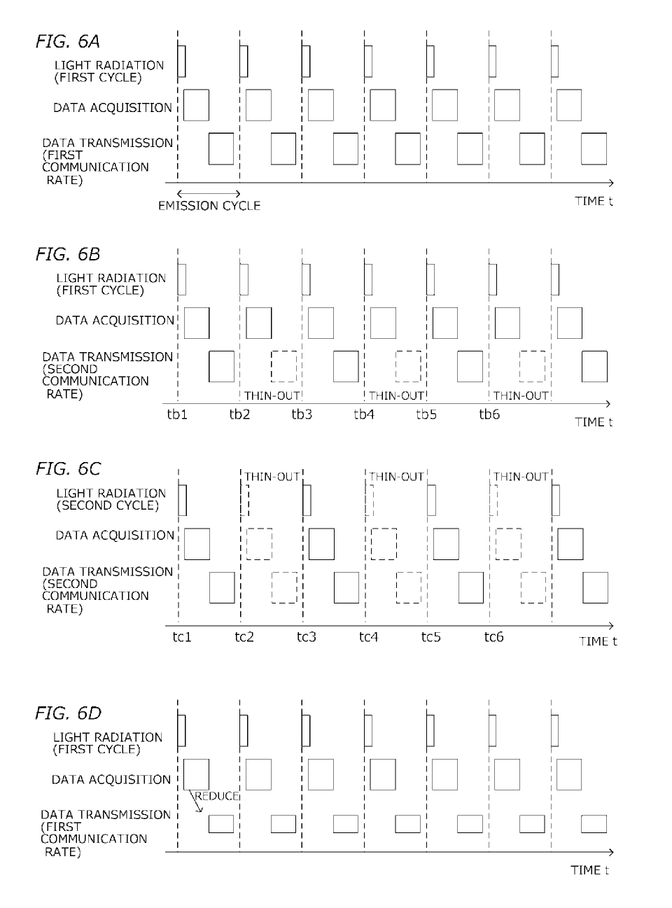

A specific embodiment of a method for transmitting photoacoustic signals according to a communication quality will be described. FIGS. 6A to 6D are timing charts of operations of a system according to the present embodiment. FIG. 6A corresponds to a case in which the communication environment is “good” and is a timing chart in which light is irradiated at the first cycle (emission cycle) to acquire photoacoustic signals and data is transmitted at the first communication rate. In a case where the communication quality is good, since all photoacoustic signals obtained by one radiation of light can be transmitted until the next radiation of light, photoacoustic images can be displayed with a high image quality and a high frame rate.

FIG. 6B illustrates a process in a case where the communication environment is “bad” and the available memory capacity is “sufficient”. In this example, similarly to FIG. 6A, radiation of light and acquisition of photoacoustic signals are performed at the first cycle (emission cycle). On the other hand, during data transmission, only the pieces of data obtained by radiation of light at timings tb1, tb3, tb5, and the like are transmitted, the other pieces of data are thinned out. All pieces of data including the data thinned out during transmission are stored in the memory 17.

FIG. 6C illustrates a process in a case where the communication environment is “bad” and the available memory capacity is “insufficient”. In this example, the driver 12 instructs the light source unit 11 to emit light at the second cycle lower than the first cycle. As a result, light is irradiated at the timings tc1, tc3, tc5, and the like only and the corresponding photoacoustic signals are acquired. Therefore, the amount of data transmitted and the amount of data stored decrease. In the case of FIG. 6C, an effect of reducing power consumption of the light source unit is obtained.

In FIGS. 6B and 6C, the determining unit decreases the amount of data transmitted per predetermined unit period by thinning out the photoacoustic signals in order to cope with deterioration in the communication quality. In this case, the image quality of the photoacoustic images does not decrease whereas the frame rate decreases. On the other hand, according to the method of FIG. 6D, it is not necessary to decrease the frame rate.

That is, in FIG. 6D, the determining unit decreases the amount of data transmitted per unit period by reducing the amount of the data of photoacoustic signals. In this example, the photoacoustic probe includes a adding circuit that sums the outputs from the signal processing unit 14. The adding circuit reduces a data amount by adding signals output from a plurality of adjacent transducers according to the control of the determining unit and adding digital signals output at a predetermined sampling intervals from the A/D converter. This addition process may use a S/N ratio improvement adding circuit and may use another adding circuit separately from the S/N ratio improvement adding circuit. The storage rate may be determined according to the available capacity of the memory 17. According to the method of FIG. 6D, since the amount of data transmitted per unit period can be reduced, even in a case where the communication quality is bad, it is possible to display photoacoustic images without decreasing the frame rate.

Although the amount of transmitted data is reduced approximately by half in the present embodiment, the reduction ratio of the amount of transmitted data can be adjusted by adjusting a thinning-out timing or a data compression method. It is also preferable that during photoacoustic measurement, a process of setting the communication rate and the storage rate in FIG. 4B is performed periodically, and in a case where the communication environment is improved and an available capacity is formed, the communication rate and the storage rate are reexamined appropriately.

The user may determine which one of the methods of FIGS. 6B to 6D will be employed in a case where the communication quality is bad or the available memory capacity is small. In this case, the user operates the input unit to select a mode in which either the frame rate or the image quality is prioritized.

In FIGS. 6A to 6D, a data transmission timing is synchronized with the timing of radiation of light and acquisition of data. Due to this, signal processing and transmission processing for photoacoustic signals, which increase due to sampling after radiation of light is not delayed, and image display with high real-time properties can be realized. However, it is not essential that radiation of light and acquisition of data is synchronized with transmission of data.

As described above, according to the present invention, in a case where a handheld photoacoustic probe and a photoacoustic apparatus body perform communication to transmit and receive data, an appropriate communication rate and an appropriate storage rate are determined on the basis of the quality of wireless communication and the data capacity of a memory inside the probe. Therefore, it is possible to set an optimal communication rate regardless of the quality of a communication environment and to store clinical data reliably.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-229202, filed on Nov. 29, 2017, which is hereby incorporated by reference herein in its entirety.

What is claimed is:

1. A photoacoustic probe comprising:
a receiving unit configured to receive photoacoustic waves generated from an object irradiated with light;
a signal processing unit configured to perform a process for outputting photoacoustic signals originating from the photoacoustic waves;
a communication unit configured to perform wireless communication with a photoacoustic apparatus body to transmit the photoacoustic signals;
a communication quality acquiring unit configured to acquire a communication quality of wireless communication which the communication unit performs;
a memory configured to store the photoacoustic signals; and
a determining unit configured to determine, according to the communication quality, a communication rate of the communication unit and whether to store the photoacoustic signals in the memory.

2. The photoacoustic probe according to claim 1, further comprising:
a capacity acquiring unit configured to acquire an available capacity of the memory, wherein
the determining unit determines, according to the available capacity, a storage rate at which data is stored in the memory.

3. The photoacoustic probe according to claim 2, wherein
the determining unit sets the communication rate to a first communication rate and determines not to store the photoacoustic signals in the memory in a case where the communication quality is equal to or higher than a threshold, and sets the communication rate to a second communication rate lower than the first communication rate in a case where the communication quality is lower than the threshold.

4. The photoacoustic probe according to claim 3, wherein
the first communication rate is a communication rate at which all the photoacoustic signals output from the signal processing unit can be transmitted.

5. The photoacoustic probe according to claim 3, wherein
the second communication rate is determined according to the acquired communication quality.

6. The photoacoustic probe according to claim 3, wherein
the second communication rate is a fixed value determined on the basis of an image quality or a frame rate in a case where a display unit of the photoacoustic apparatus body displays photoacoustic images based on the photoacoustic signals.

7. The photoacoustic probe according to claim 2, wherein
the determining unit sets the storage rate to a first storage rate in a case where the available capacity is larger than a total amount of the photoacoustic signals output from the signal processing unit, and sets the storage rate to a second storage rate lower than the first storage rate in a case where the available capacity is smaller than the total amount.

8. The photoacoustic probe according to claim 3, wherein
the determining unit performs a thinning-out process on the photoacoustic signals in a case where the communication rate is the second communication rate.

9. The photoacoustic probe according to claim 3, wherein
the determining unit performs an addition process on the photoacoustic signals in a case where the communication rate is the second communication rate.

10. The photoacoustic probe according to claim 1, wherein
the communication quality acquiring unit acquires the communication quality on the basis of a communication speed of the wireless communication.

11. The photoacoustic probe according to claim 2, further comprising:
a notifying unit configured to issue a notification of the communication quality and the available capacity.
